(19) United States
(12) Patent Application Publication
(10) Pub. No.: US 2023/0070840 A1
(43) Pub. Date: Mar. 9, 2023

(54) PREDICTING PATIENT SURVIVAL

(71) Applicant: AROCELL AB, Uppsala (SE)

(72) Inventors: Staffan ERIKSSON, Lidingö (SE); Martin SHAW, Kirkkonummi (FI); Teuvo TAMMELA, Tampere (FI); Teemu MURTOLA, Kangasala (FI)

(21) Appl. No.: 17/794,286

(22) PCT Filed: Jan. 29, 2021
(86) PCT No.: PCT/SE2021/050060

§ 371 Date: Jul. 21, 2022

(30) Foreign Application Priority Data
Jan. 30, 2020 (SE) 2050091-4

(51) Int. Cl. G01N 33/574; G01N 33/543

(52) U.S. Cl. CPC G01N 33/57434; G01N 33/54386; G01N 2800/52; G01N 2333/912

(57) ABSTRACT

A method for predicting patient survival comprises determining a level of STK1 (serum thymidine kinase 1) material in a body sample from a patient diagnosed with prostate cancer using an antibody or a fragment thereof specifically binding to a serum form of human TK1. The method also comprises predicting survival of the patient based on the determined level of STK1 material in the body sample.

### TECHNICAL FIELD

The present invention generally relates to measurement of thymidine kinase 1 (TK1) and in particular to predicting patient survival based on measured serum TK1 levels.

### BACKGROUND

Thymidine kinase 1 (TK1) (EC 2.7.1.21), also referred to as 2′-deocythymidine kinase or ATP-thymidine 5′-phosphotransferase, is an enzyme involved in DNA precursor synthesis. TK1 phosphorylates thymidine to allow incorporation into DNA. Expression of TK1 is a marker of active cellular proliferation with low intracellular concentration during G0/G1 phase of the cell cycle, and increasing during the S/G2 phases.

A form of TK1 is present at high levels also in sera and plasma from humans and animals with malignant tumors. Therefore, serum TK1 activity measurements have been used for monitoring and for prognostic purpose in several different malignant diseases, but primarily in case of leukemia and lymphoma.

Furthermore, TK1 is the only proliferation marker that can be determined in the blood and it is likely to provide a large clinical benefit if available as a routine laboratory test.

The serum TK1 activity has been measured using a radioactive substrate ¹²⁵I-dUrd (the PROLIFIGEN® TK-REA, DiaSorin Inc) for several decades, but this radio-enzymatic assay has had limited use and preferentially in case of malignant hematologic malignancies. A non-radiometric TK1 activity assay (TK LIAISON® assay, DiaSorin Inc.) has been available in recent years. This is a sensitive and robust assay and has provided clinically valuable information in humans and dogs mainly in hematologic malignancies, particularly for monitoring therapy and predicting relapse.

During the last 15 years antibodies against human TK1 have been available and enabled the determination of the TK1 protein levels, in contrast to TK1 activity, both in hematologic as well as in solid tumor diseases, such as breast carcinomas, and several other forms of solid and hematologic tumors.

One type of TK1 protein determination relies on a dot blot procedure based on anti-TK1 antibodies produced against the C-terminal part of TK1. The main reason for choosing this strategy for antibody production is that the C-terminal region is involved in the cell cycle regulation of TK1. It contains a recognition sequence for initiating the degradation of TK1 during mitosis and it has been assumed that this is an exposed region to which it could be possible to generate antibodies. Although the dot blot assay has been used successfully in a number of studies, a major limitation is that it is not a routine method in clinical laboratory practice.

AroCell TK 210 ELISA is a quantitative immunoassay kit for the determination of TK1 in human blood. The ELISA format is simple and robust, requires no special instrumentation to perform and can easily be incorporated into standard laboratory processes. The AroCell TK 210 ELISA is not only a fast and simple way to monitor TK1 from blood samples but delivers reproducible results you can trust using standard equipment in clinical chemistry.

Jagarlamudi et al., Breast and prostate cancer patients differ significantly in their serum Thymidine kinase 1 (TK1) specific activities compared with those hematological malignancies and blood donors: implications of using serum TK1 as a biomarker, BMC Cancer (2015), 15: 66 discloses that there are differences in the specific activities and the composition of active and inactive subunits of TK1 in sera from patients with hematological malignancies compared to those with breast and prostate cancer.

Li et al., Serum thymidine kinase 1 is associated with Gleason score of patients with prostate carcinoma, Oncology Letters (2018), 16(5): 6171-6180 discloses that serum TK1 concentration is a more reliable prognostic biomarker than total prostate-specific antigen (PSA) according to Gleason scores in screening of benign prostatic hyperplasia (BPH) or prostate malignancy.

Jagarlamudi et al., AroCell TK 210 ELISA for determination of TK1 protein: age-related reference ranges and comparison with other TK1 assays, Biotechniques (2020) 68(6): 335-342 discloses that TK 210 ELISA analysis of sera from prostate and breast cancer patients had significantly higher TK1 protein levels compared to those in sera from healthy blood donors.

Jagarlamudi et al., The combination of AroCell TK 210 ELISA with Prostate Health Index of prostate-specific antigen density can improve the ability to differentiate prostate cancer from noncancerous conditions, The Prostate (2019), 79(8): 856-863 discloses that serum TK1 as measured by AroCell TK 210 ELISA is significantly higher in patients with prostate cancer than with benign urological conditions. The serum TK1 was correlated to Prostate Health Index but not to the Gleason score.

### SUMMARY

It is a general objective of the invention to predict patient survival of patients diagnosed with prostate cancer.

This and other objectives are met by embodiments as disclosed herein.

The present invention is defined in the independent claim. Further embodiments of the invention are defined in the dependent claims.

An aspect of the invention relates to a method for predicting patient survival. The method comprises determining a level of serum TK1 (STK1) material in a body sample from a patient diagnosed with prostate cancer using an antibody or a fragment thereof specifically binding to a serum form of human TK1. The method also comprises predicting survival of the patient based on the determined level of STK1 material in the body sample.

The present invention enables identification of prostate cancer patients having a poor predicted survival rate at the time of prostate cancer diagnosis. Those patients can thereby be selected for a more aggressive anti-cancer treatment or a more frequent patient surveillance.

### BRIEF DESCRIPTION OF THE DRAWINGS

The embodiments, together with further objects and advantages thereof, may best be understood by making reference to the following description taken together with the accompanying drawings, in which:

FIG. 1 illustrates prostate cancer-specific survival by STK1 level at diagnosis. Study population of 40 and 43 men with M0 and M1 prostate cancer at diagnosis, respectively. Calculated using Cox regression model adjusted for age and PSA at diagnosis, clinical T-stage and M-stage.

FIGS. 2A to 2D illustrate prostate cancer-specific survival among 43 men with de novo metastatic disease, stratified by TK1 quartile, (A) age-adjusted, (B) adjusted for ISUP Gleason grade group, (C) adjusted for PSA at diagnosis and (D) adjusted for clinical T-stage.

FIG. 3 illustrates prostate cancer (PCa) death random forest classification error estimates for mean prediction error and class-specific prediction errors for alive and PCa death, and empirically estimated 95% confidence intervals (CI), for all models on the x-axis. The classifiers used in each model are written on the right-hand side of the image, on the y-axis.

### DETAILED DESCRIPTION

The present invention generally to measurement of TK1 and in particular to predicting patient survival based on measured serum TK1 (STK1) levels.

Previous studies as referred to in the background section have suggested that serum TK1 expression could be used as biomarker for presence of prostate cancer. Experimental data as presented herein shows that TK1 expression can be used as a biomarker for the risk of prostate cancer progression after diagnosis, and in particular that STK1 levels measured in a body sample of a subject or patient diagnosed with prostate cancer, in particular metastatic prostate cancer, can be used as a predictor of survival after cancer diagnosis.

This means that STK1 levels measured in a body sample of a patient diagnosed with prostate cancer can be used in prostate cancer risk stratification when selecting optimal treatment and surveillance schedules for prostate cancer patients. Hence, prostate cancer patients with high levels of STK1 levels have a comparatively lower predicted survival as compared prostate cancer patients with comparatively lower STK1 levels. The high risk patients could therefore be selected for more frequent surveillance and/or more aggressive cancer treatment as compared to those prostate cancer patients predicted to have high survival.

An aspect of the invention relates to a method for predicting patient survival. The method comprises determining a level of STK1 material in a body sample from a patient diagnosed with prostate cancer using an antibody or a fragment thereof specifically binding to a serum form human TK1. The method also comprises predicting survival of the patient based on the determined level of STK1 material in the body sample.

Thus, the invention is based on the unexpected finding that STK1 levels as measured from patients suffering from and diagnosed with prostate cancer correlates with survival of the prostate cancer patients. In other words, STK1 levels can be used as a predictor for the survival of the diagnosed patients in terms of predicted or estimated remaining life span. Generally, the higher STK1 level as measured in the body sample from a prostate cancer patient the poorer predicted survival. Hence, a comparatively high STK1 level correlates with a high hazard ratio (HR) of mortality.

In an embodiment, the method also comprises obtaining the body sample from the patient.

The body sample is preferably a body fluid sample comprising STK1 material. Non-limiting, but preferred, examples of such body fluid samples include a serum sample, a plasma sample, a blood sample, a synovial fluid sample, a lymphatic fluid sample, a urine sample and a saliva sample, preferably a serum sample or a plasma sample. Although body fluid samples are preferred body samples, the embodiments are not limited thereto and may also use solid body samples, such as biopsies from the prostate.

According to the invention, determining the level of TK1 material comprises determining a level of serum TK1 (STK1) material in a body sample, preferably a serum or plasma sample, obtained from the patient using an antibody or a fragment thereof specifically binding to a serum form of human TK1.

TK1 in humans are present in various forms depending on the presence of certain molecules, e.g., presence or absence of adenosine triphosphate (ATP); depending on the concentration of the protein, i.e., high or low concentration; depending on the type of the protein, i.e., native or recombinant TK1; and depending on the site of the protein, i.e., in serum or cytoplasma.

Generally, cytosolic and recombinant human TK1 occurs as tetramers in the presence of ATP or at high concentration, and as dimers in the absence of ATP or at low concentration. The tetramer form of cytosolic and recombinant human TK1 has high TK1 activity whereas the dimer form has lower TK1 activity. Cytosolic TK1, also referred to as cellular TK1, is TK1 present inside cells and can be isolated from such cells.

Human STK1, in clear contrast, can be in the form of high molecular weight complexes, such as oligomers or comprising such oligomers, having TK1 activity and dimer and tetramer forms having very low or even lacking TK1 activity. The oligomerization seems to be related to the formation of disulfide cross linking occurring in the blood. STK1 is found in the blood of a patient and can thereby be determined in, among others, a blood sample, a plasma sample or a serum sample.

STK1 material as used herein refers to STK1 in its various forms, such as dimers, tetramers, oligomers and complexes comprising STK1. The STK1 material is a serum TK1 material, i.e., a STK1 material present in blood, blood plasma or serum in patient. The STK1 material may then comprise STK1 in the above mentioned forms, such as dimers, tetramers, oligomers and complexes comprising STK1. STK1 material also includes complexes with at least one TK1 protein unit and other molecules and/or macromolecules.

In the art, various gene expressions arrays have been proposed to determine the TK1 mRNA transcripts in cancer cell samples and biopsies, including prostate cell samples and prostate biopsies. As mentioned above, TK1 is available in various forms in subjects, including cytosolic TK1 and serum TK1. Gene expression arrays determining TK1 mRNA transcripts from such cell or biopsy samples are mainly assaying TK1 mRNA transcripts of cytosolic TK1 present in cancer cells. Hence, such gene expression arrays cannot be used to determine the level of STK1 material in a subject.

In an embodiment, determining the level of STK1 material comprises contacting the body sample with the antibody or the fragment thereof. This embodiment also comprises measuring an amount of antibody or fragment thereof bound to the STK1 material.

Contacting the body sample with the antibody or the fragment thereof may be achieved by adding the antibody or the fragment thereof to the body sample and incubating the body sample with the antibody or the fragment thereof. The antibody or the fragment thereof thereby binds to the STK1 material forming a complex between the antibody or the fragment thereof and the STK1 material. In such an embodiment, measuring the amount of antibody or fragment bound to the STK1 material can include measuring or quantifying the complex between the antibody or the fragment thereof and the STK1 material to thereby measure or quantify the amount of antibody or fragment bound to the STK1 material.

In an embodiment, the method also comprises correlating the measured amount of antibody or fragment bound to the STK1 material to a level of STK1 material. This may be performed using a pre-defined correlation between measured amount of antibody or fragment bound to a reference TK1 material and concentration of the reference TK1 material. A typical reference TK1 material that can be used when generating such a pre-defined correlation is recombinant human TK1.

The pre-defined correlation may, thus, be generated by adding the antibody or the fragment thereof to different samples comprising different concentrations of the reference TK1 material, preferably recombinant human TK1. The amount of antibody or fragment bound to the reference TK1 material, preferably recombinant human TK1, is then measured in the different samples to thereby get a standard curve, function or relationship between concentration of reference TK1 material, preferably recombinant human TK1, and the measured amount of antibody or fragment bound to the reference TK1 material, preferably recombinant human TK1.

This pre-defined correlation, such as standard curve, function or relationship, can then be used to map or convert the measured amount of antibody or fragment bound to the STK1 material in the body sample to a concentration of the STK1 material in the body sample.

It is generally preferred if the same type of antibody or fragment thereof is used for generating the pre-defined correlation as for determining a level of STK1 material in a body sample from the patient diagnosed with prostate cancer. Hence, in a preferred embodiment, the antibody or the fragment thereof is capable of specifically binding to not only the serum form of human TK1 but also to the reference TK1 material, preferably recombinant human TK1.

In an embodiment, the body sample is processed prior to or during the incubation of the body sample with the antibody or the fragment thereof. This sample processing may be used to stabilize selected STK1 forms in the body sample and/or to break larger STK1 complexes or oligomers into smaller complexes or multimers.

Hence, in an embodiment, a sample dilution or pretreatment buffer is added to the body sample prior to or in connection with adding the antibody or the fragment thereof to the body sample, preferably prior to adding the antibody or the fragment thereof to the body sample.

In an embodiment, the sample dilution buffer comprises ATP, preferably in a concentration selected within an interval of from 0.5 mM up to 50 mM, such as from 0.5 mM up to 20 mM or from 1.5 mM up to 50 mM. As previously described herein, ATP stabilizes the tetramer form of TK1, which has high enzymatic TK1 activity.

In another embodiment, the sample dilution buffer comprises a reducing agent. The reducing agent may then break disulfide cross links in larger STK1 complexes and oligomers to obtain smaller STK1 forms, such as tetramers. Various reducing agents capable of breaking disulfide bonds can be used according to the embodiments including, but not limited to, dithioerythritol (DTE), dithiothreitol (DTT), dithiobutylamin (DTBA), tris(2-carboxyethyl)phosphine) (TCEP), and combinations thereof. The amount of the reducing agent is typically selected within an interval of from 0.1 mM up to 10 mM.

The sample dilution buffer may, in an embodiment, comprise both ATP and a reducing agent.

The level of STK1 material is, in an embodiment, determined using the antibody or the fragment thereof specifically binding to the serum form of human TK1 in the body sample taken from the patient in connection with diagnosing the patient with prostate cancer. Hence, in a preferred embodiment, the body sample is preferably taken at the time of diagnosis or at least shortly following the point in time at which the patient was diagnosed with prostate cancer or at least suspected to suffer from prostate cancer.

In a particular embodiment, the level of STK1 material is determined in the body sample from a patient diagnosed with metastatic prostate cancer using the antibody or the fragment thereof specifically binding to the serum form of human TK1. Hence, in this particular embodiment, the patient is diagnosed with metastatic prostate cancer. In a particular embodiment, the patient is diagnosed with M1 stage of prostate cancer.

Generally, M0 stage means that the prostate cancer has not spread to other parts of your body. M1 stage means the cancer has spread to other parts of the body outside the pelvis. Sometimes the M1 stage is split into M1a—cancer cells in lymph nodes outside the pelvis, M1b—cancer cells in the bone and M1c—cancer cells in other parts of the body, such as in the liver, brain and/or lungs.

In an embodiment, the method comprises estimating a hazard ratio (HR) for the patient based on the determined level of STK1 material in the body sample. In such an embodiment, predicting survival of the patient comprises predicting survival of the patient based on the estimated HR. Experimental data as presented herein shows that prostate cancer patients with high levels of STK1 material in their body samples have high HR and poor predicted survival, whereas prostate cancer patients with comparatively lower levels of STK1 material in their body samples have comparatively lower HR and better predicted survival in terms of predicted survival span following diagnosis of prostate cancer.

In an embodiment, the method comprises comparing the determined level of STK1 material in the body sample with a threshold value. In such an embodiment, predicting survival of the patient comprises predicting poor survival of the patient if the determined level of STK1 material in the body sample exceeds the threshold value and otherwise predicting good survival of the patient.

STK1 level measured at the diagnosis of prostate cancer for patients with M0 prostate cancer at diagnosis had an interquartile range (IQR), also called the midspread, middle 50%, or H-spread, of 0.15-0.41 μg/L (0.152-0.412 μg/L) with a median value of 0.23 μg/L (0.228 μg/L). The corresponding IQR for patients with M1 prostate cancer at diagnosis was 0.33-0.82 μg/L (0.332-0.821 μg/L) with a median value of 0.61 μg/L (0.611 μg/L).

In an embodiment, the threshold value is selected within an interval of from 0.15 μg/L to 0.45 μg/L, preferably within an interval of from 0.175 μg/L to 0.40 μg/L, such as within an interval of from 0.20 μg/L to 0.30 μg/L, and more preferably within an interval of from 0.20 μg/L to 0.25 μg/L, such as 0.23 μg/L. This threshold value is particular suitable for prostate cancer patients diagnosed with M0 prostate cancer.

In another embodiment, the threshold value is selected within an interval of from 0.30 μg/L to 0.90 μg/L, preferably within an interval of from 0.40 μg/L to 0.80 μg/L, such as within an interval of from 0.50 μg/L to 0.70 μg/L, and more preferably within an interval of from 0.60 μg/L to 0.65 μg/L, such as 0.61 μg/L. This threshold value is particular suitable for prostate cancer patients diagnosed with M1 prostate cancer.

In a further embodiment, the threshold value is selected within an interval of from 0.25 μg/L to 0.45 μg/L, preferably within an interval of from 0.275 μg/L to 0.425 μg/L, such as within an interval of from 0.30 μg/L to 0.40 μg/L, and more preferably within an interval of from 0.325 μg/L to 0.375 μg/L, such as about 0.35 μg/L, for instance 0.348 μg/L. This threshold value can be used for prostate cancer patients diagnosed with prostate cancer regardless of the cancer stage.

In yet another embodiment, the threshold value is selected within an interval of from 0.25 to 0.75 μg/L, preferably within an interval of from 0.30 to 0.65 μg/L, and more preferably within an interval of from 0.325 and 0.375 μg/L.

It is also possible to use multiple, i.e., at least two, different threshold values in predicting survival of the prostate cancer patients. For instance, a first threshold value and a second threshold value could be used to differentiate between patients with a good survival rate, having a determined level of STK1 material in the body sample below, or equal to or below, the first threshold value, patients with a medium or intermediate survival rate, having a determined level of STK1 material in the body sample equal to or above, or above, the first threshold value but below, or equal to or below, the second threshold value and patients with poor survival rate, having a determined level of STK1 material in the body sample equal to or above, or above, the second threshold value.

In an embodiment, the first threshold value is selected within an interval of from 0.15 μg/L to 0.45 μg/L, preferably within an interval of from 0.175 μg/L to 0.40 μg/L, such as within an interval of from 0.20 μg/L to 0.30 μg/L, and more preferably within an interval of from 0.225 μg/L to 0.25 μg/L, such as 0.24 μg/L. In this embodiment, the second threshold value is selected within an interval of from 0.30 μg/L to 0.90 μg/L, preferably within an interval of from 0.40 μg/L to 0.80 μg/L, such as within an interval of from 0.45 μg/L to 0.70 μg/L, and more preferably within an interval of from 0.50 μg/L to 0.60 μg/L, such as 0.54 μg/L or 0.537 μg/L, with the proviso that the second threshold value is larger than the first threshold value.

In an embodiment, the method also comprises determining prostate-specific antigen (PSA) in a body sample from the patient. In this embodiment, predicting survival of the patient comprises predicting survival of the patient based on the determined level of STK1 material in the body sample and based on the determined PSA in the body sample.

PSA, also known as gamma-seminoprotein or kallikrein-3 (KLK3), is a glycoprotein enzyme encoded in humans by the KLK3 gene. PSA is present in small quantities in the serum of men with healthy prostates, but is often elevated in the presence of prostate cancer or other prostate disorders. PSA is not uniquely an indicator of prostate cancer, but may also detect prostatitis or benign prostatic hyperplasia.

PSA can be determined in the same body sample taken from the patient as the STK1 material. Alternatively, PSA can be determined in another body sample taken from the patient. The above discussed examples of body samples also apply to PSA. For instance, PSA can be determined in a body fluid sample taken from the patient, in particular a blood sample, a plasma sample or a serum sample.

In an embodiment, the method also comprises determining International Society of Urological Pathology (ISUP) Gleason grade group for the patient. In this embodiment, predicting survival of the patient comprises predicting survival of the patient based on the determined level of STK1 material in the body sample and based on the determined ISUP Gleason grade group.

Gleason score is given after a pathologist has examined, under a microscope, cancerous tissue obtained from a biopsy. The cells identified are given a grade number from 1 to 5, depending on the abnormality of the cells, 1 being the lowest, 5 the highest. The grades of the two most common patterns are added together to give a score from 2 to 10. The higher the score the more aggressive and fast-growing the cancer. Scores totaling 5 or less are insignificant and are not reported. ISUP Gleason grade group, also referred to as ISUP grade group, is a prostate cancer grading system released by the International Society of Urological Pathology (Am J Surg Pathol (2020) 44(8): e87-e99). The mapping between ISUP Gleason grade group and Gleason score is shown in Table 1.

TABLE 1
Mapping between ISUP Gleason
grade group and Gleason score

| | ISUP | | |
|---|---|---|---|
| | Gleason | | |
| | grade | Gleason | |
| Risk group | group | score | |
| Low | 1 | <6 | |
| Intermediate Favorable | 2 | 7 | (3 + 4) |
| Intermediate Unfavorable | 3 | 7 | (4 + 3) |

| | | |
|---|---|---|
| High | 4 | 8 |
| | 5 | 9-10 |

As is shown in Table 1, there is a relationship between ISUP Gleason grade group and Gleason score. Hence, determining ISUP Gleason grade group for the patient and predicting survival of the patient based on the determined level of STK1 material in the body sample and based on the determined ISUP Gleason grade group can alternatively be performed by determining Gleason score for the patient and predicting survival of the patient based on the determined level of STK1 material in the body sample and based on the determined Gleason score.

In a particular embodiment, the method comprises determining PSA in a body sample from the patient. The method also comprises, in this embodiment, determining ISUP Gleason grade group for the patient. In this embodiment, predicting survival of the patient comprises predicting survival of the patient based on the determined level of STK1 material in the body sample, based on the determined PSA in the body sample and based on the determined ISUP Gleason grade group.

The antibody or the fragment thereof specifically binds to the STK1 material, and in particular binds specifically to the serum form of the TK1 protein.

The specificity of an antibody or a fragment thereof can be determined based on affinity and/or avidity. The affinity, represented by the equilibrium constant for the dissociation of an antigen with the antibody or the fragment thereof (K_{d}), is a measure for the binding strength between an antigenic determinant and an antigen-binding site on the antibody or the fragment thereof. The lesser the value of K_{d}, the stronger the binding strength between the antigenic determinant and the antibody or the fragment thereof. Alternatively, the affinity can also be expressed as the affinity constant (Kₐ), which is 1/K_{d}. As will be clear to the skilled person, affinity can be determined in a manner known per se, depending on the specific antigen of interest.

Avidity is the measure of the strength of binding between an antibody or a fragment thereof and the pertinent antigen. Avidity is related to both the affinity between an antigenic determinant and its antigen binding site on the antibody or the fragment thereof and the number of pertinent binding sites present on the antibody or the fragment thereof.

Typically, antibodies will bind to their antigen with a dissociation constant (K_{d}) of 10⁻⁵ to 10⁻¹² moles/liter (M) or less, and preferably 10⁻⁷ to 10⁻¹² M or less and more preferably 10⁻⁸ to 10⁻¹² M, i.e. with an association constant (Kₐ) of 10⁵ to 10¹² M⁻¹ or more, and preferably 10⁷ to 10¹² M⁻¹ or more and more preferably 10⁸ to 10¹² M⁻¹.

Generally, any K_{d} value greater than 10⁻⁴ M (or any Kₐ value lower than 10⁴ M⁻¹) is generally considered to indicate non-specific binding. Preferably, an antibody or a fragment thereof will bind to the STK1 material with an affinity less than 500 nM, preferably less than 200 nM, more preferably less than 10 nM, such as less than 5 nM or even lower, such as 1 nM or lower.

Specific binding of an antibody or a fragment thereof to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

In an embodiment, the antibody is a monoclonal antibody, i.e., a monoclonal anti-TK1 antibody. In another embodiment, the antibody is a polyclonal antibody, i.e., a polyclonal anti-TK1 antibody.

In an embodiment, the antibody or the fragment thereof has specificity for an epitope or peptide consisting of an amino acid sequence from the C-terminal region of TK1, preferably of human TK1.

The peptide is preferably selected from a portion of TK1 ranging from amino acid position 200 to the end of the TK1, i.e., amino acid position 234 in humans (SEQ ID NO: 28). In a particular embodiment, the peptide is selected from a portion of the TK1 protein ranging from amino acid position 205, preferably 210 to amino acid position 230, preferably 225.

The peptide is preferably an N-mer, wherein N is an integer within a range of from 8 up to 20, preferably within a range of from 10 up to 15. The peptide preferably consists of N consecutive amino acids in the C-terminal region of the TK1 protein.

In an embodiment, the peptide consists of the following amino acid sequence GEAVAARKLF (SEQ ID NO: 1). In another embodiment, the peptide consists of the following amino acid sequence NCPVPGKPGE (SEQ ID NO: 2). In a further embodiment, the peptide consists of the following amino acid sequence PVPGKPGEAV (SEQ ID NO: 3). In yet another embodiment, the peptide consists of the following amino acid sequence NCPVPGKPGEAV (SEQ ID NO: 4).

A monoclonal antibody having specificity for an epitope consisting of GEAVAARKLF (SEQ ID NO: 1) has a variable heavy (VH) domain complementarity determining region 1 (CDR1) having amino acid sequence DYEMH (SEQ ID NO: 5), a VH domain CDR2 having amino acid sequence AIHPGYGGTAYNQKFKG (SEQ ID NO: 6), a VH domain CDR3 having amino acid sequence FITKFDY (SEQ ID NO: 7), a variable light (VL) domain CDR1 having amino acid sequence KSSQSLLDSDGKTFLN (SEQ ID NO: 8), a VL domain CDR2 having amino acid sequence LVSKLDS (SEQ ID NO: 9) and a VL domain CDR3 having amino acid sequence WQGTHFPWT (SEQ ID NO: 10).

A monoclonal antibody having specificity for the epitopes NCPVPGKPGE (SEQ ID NO: 2), PVPGKPGEAV (SEQ ID NO: 3) and NCPVPGKPGEAV (SEQ ID NO: 4) has a VH domain CDR1 having amino acid sequence DYEMH (SEQ ID NO: 5), a VH domain CDR2 having amino acid sequence AILPGSGGTAYNQKFKG (SEQ ID NO: 11), a VH domain CDR3 having amino acid sequence LITTFDY (SEQ ID NO: 12), a VL domain CDR1 having amino acid sequence KSSQSLLDSDGKTYLN (SEQ ID NO: 13), a VL domain CDR2 having amino acid sequence LVSKLDS (SEQ ID NO: 9), and a VL domain CDR3 having amino acid sequence WQGTHFPWT (SEQ ID NO: 10).

In another embodiment, the antibody or the fragment thereof has specificity for a conformation dependent epitope of human TK1. A monoclonal antibody having specificity for such a conformation dependent epitope has a VH domain CDR1 having amino acid sequence SGYSWH (SEQ ID NO: 14), a VH domain CDR2 having amino acid sequence YIHYSGSTTYNPSLKG (SEQ ID NO: 15), a VH domain CDR3 having amino acid sequence WGTGHWYFDV (SEQ ID NO: 16), a VL domain CDR1 having amino acid sequence RSSTGAVTTTNYAN (SEQ ID NO: 17), a VL domain CDR2 having amino acid sequence GTNNRVP (SEQ ID NO: 18), and a VL domain CDR3 having amino acid sequence ALWYSNHWV (SEQ ID NO: 19).

The above-three presented examples of monoclonal anti-TK1 antibodies that can be used according to the embodiments are further disclosed in WO 2015/094106, the teaching of which regarding monoclonal anti-TK1 antibodies is incorporated herein by reference.

Hence, in an embodiment, the monoclonal antibody or the fragment thereof is selected from the group consisting of a monoclonal antibody or a fragment thereof having specificity for GEAVAARKLF (SEQ ID NO: 1) of human TK1, a monoclonal antibody or a fragment thereof having specificity for at least one of NCPVPGKPGE (SEQ ID NO: 2), PVPGKPGEAV (SEQ ID NO: 3) and NCPVPGKPGEAV (SEQ ID NO: 4) of human TK1, and a monoclonal antibody or a fragment thereof having specificity for a conformation dependent epitope of human TK1.

In another embodiment, the antibody or the fragment thereof has specificity for an epitope or peptide consisting of KPGEAVAARKLFAPQ (SEQ ID NO: 20). At least one additional amino acid, such as a cysteine residue, may be added to the N-terminal or C-terminal, preferably the N-terminal, of the peptide for use as coupling to other molecules, such as carrier proteins.

An antibody having specificity for this epitope is further disclosed in WO 95/29192, the teaching of which regarding anti-TK1 antibodies is incorporated herein by reference.

In a further embodiment, the antibody or fragment thereof has specificity for an epitope or peptide consisting of an amino acid sequence from an active site of TK1. The peptide is preferably selected from a portion of TK1 ranging from amino acid position 150 to amino acid position 190 in human TK1. In a particular embodiment, the peptide is selected from a portion of TK1 ranging from amino acid position 155, preferably 160 and more preferably 161, to amino acid position 185, preferably 183.

The peptide is preferably an M-mer, wherein M is an integer within a range of from 10 up to 40, preferably within a range from 20 up to 30 and more preferably 23 or 24. The peptide preferably consists of M consecutive amino acids in the active site of the TK1 protein.

At least one additional amino acid, such as a cysteine residue, may be added to the N-terminal or C-terminal, preferably the N-terminal, of the peptide for use as coupling to other molecules, such as carrier proteins.

In an embodiment, the peptide consisting of an amino acid sequence from the active site of TK1 has an amino acid sequence corresponding to amino acid positions 161 to 183 in human TK1, i.e., has amino acid sequence of AYTKRLGTEKEVEVIGGADKYHS (SEQ ID NO: 21).

An antibody having specificity for this epitope is further disclosed in WO 2008/142664, the teaching of which regarding anti-TK1 antibodies is incorporated herein by reference.

In a further embodiment, the antibody or the fragment thereof is a monoclonal antibody or a fragment thereof as disclosed in WO 2019/201901, the teaching of which regarding monoclonal anti-TK1 antibodies is incorporated herein by reference.

For instance, the monoclonal antibody could be mAb 6C6, mAb 4H4 or mAb 23C11.

mAb 6C6 VH domain (SEQ ID NO: 22):
METGLRWLLLVAVLKGVQCQEQLEESGGDLVKPEGSLTLTCTASRFSFS
SSYWICWRQAPGKGLEWIACIYAGDSGSSYYASWAKGRFTVSKTSSTTV
TLQTTSLTAADTATYFCARASVGAAYDYFALWGPGTLVTVSSGQPKAPS
VFPLAPCCGDTPSSTVTLGCLVKGYLPEPVTVTWNSG
mAb 6C6 VL domain (SEQ ID NO: 23):
MDTRAPTQLLGLLLLWLPGARCALVMTQTPASVEAAMGGTVTIKCQASE
DVSSHLAWYQQRPGQPPKLLIYGASDLASGVPSRFTGSGSGTQFTLAIS
DLECADAATYYCQGYYYISDSPYVFGGGTEWKGDPVAPTVLIFPPAADQ
VATGTVTIVCVANKYFPDVTVTWEVDGTTQTTGIENSKTPQNSADCTYN
LSSTLTLTSTQYNSHKEYTCKVTQGTTSWQSFNRGDC
mAb 4H4 VH domain (SEQ ID NO: 24):
METGLRWLLLVAVLKGVQCQSLEESGGGLVQPEGSLTLTCTASGFSFSS
GYDMCWVRQTPGKGLEWIACISVDSDGVTYYASWAKGRFTISKTSSTTV
TLQMTSLTAADTATYFCARGYESSSGVYIPYFTLWGPGTLVTVSSGQPK
APSVFPLAPCCGDTPSSTVTLGCLVKGYLPEPVTVTWNSG
mAb 4H4 VL domain (SEQ ID NO: 25):
MDMRAPTQLLGLLLLWLPGARCADIVLTQTPASVEAAVGGTVTIKCQAS
QSIYSYLAWYQHKPGQPPKLLIYKASTLASGVPSRFKGSGSGTEYTLTI
SDLECADAATYYCQHYYYSSTSGGGVFGGGTEVVVKGDPVAPTVLIFPP
AADQVATGTVTIVCVANKYFPDVTVTWEVDGTTQTTGIENSKTPQNSAD
CTYNLSSTLTLTSTQYNSHKEYTCKVTQGTTSWQSFNRGDC
mAb 23C11 VH domain (SEQ ID NO: 26):
METGLRWLLLVAVLKGVQCQSLEESGGRLVTPGTPLTLTCTASGFSLSN
YYMSWVRQAPGKGLEWIGIIYGDDNTYCANWTKGRFTISKTSTTVDLTI
TSPTTEDTATYFCARGPDYIAAKMDIWGPGTLVTVSLGQPKAPSVFPLA
PCCGDTPSSTVTLGCLVKGYLPEPVTVTWNSG
mAv 23C11 VL domain (SEQ ID NO: 27):
MDTRAPTQLLGLLLLWLPGARCDVVMTQTPASVEAAVGGTVTIKCQASQ
SISGYLSWYQQKPGQRPKLLIYRASTLESGVPSRFKGSGSGTEFTLTIS
DLECADAATYYCQCTYGSSTFSSYGNAFGGGTEVWKGDPVAPTVLIFPP
AADQVATGTVTIVCVANKYFPDVTVTWEVDGTTQTTGIENSKTPQNSAD
CTYNLSSTLTLTSTQYNSHKEYTCKVTQGTTSVVQSFNRGDC

In an embodiment, the level of STK1 material in the body sample is determined using a kit. The kit preferably comprises a first antibody or a first fragment thereof and a second antibody or a second fragment thereof. The first and second antibodies can be selected from the above described illustrative examples of monoclonal and polyclonal anti-TK1 antibodies.

In a particular embodiment, the kit comprises a first monoclonal antibody or a first fragment thereof having specificity for an epitope selected from the group consisting of i) GEAVAARKLF (SEQ ID NO: 1) of human TK1, ii) at least one of NCPVPGKPGE (SEQ ID NO: 2), PVPGKPGEAV (SEQ ID NO: 3) and NCPVPGKPGEAV (SEQ ID NO: 4) of human TK1, and iii) a conformation dependent epitope of human TK1. The kit also comprises a second monoclonal antibody or a second fragment thereof having specificity for an epitope selected from the group consisting of i) GEAVAARKLF (SEQ ID NO: 1) of human TK1, ii) at least one of NCPVPGKPGE (SEQ ID NO: 2), PVPGKPGEAV (SEQ ID NO: 3) and NCPVPGKPGEAV (SEQ ID NO: 4) of human TK1, and iii) a conformation dependent epitope of human TK1.

In an embodiment, the first antibody or the first fragment thereof is a so-called capture antibody immobilized to a support or intended to be immobilized to the support and the second antibody or the second fragment thereof is a so-called detection antibody. In another embodiment, the second antibody or the second fragment thereof is the capture antibody immobilized to the support or intended to be immobilized to the support, whereas the first antibody or the first fragment thereof is used as detection antibody.

In an embodiment, the first and second antibodies or the first and second fragments have specificities for different epitopes in the STK1 material.

In another embodiment, the first and second antibodies or the first and second fragments have specificities for the same epitope in the STK1 material. This is possible since the same epitope may be present at multiple copies in the high molecular weight complexes of multiple TK1 protein units. Thus, the STK1 material may be a multivalent complex of multiple, i.e., at least two, TK1 protein units. In fact, the same type of antibody or fragment thereof could be used as the first and second antibody or first and second fragment.

In an embodiment, one of the first and the second antibody or the first and second fragment has specificity for a peptide consisting of an amino acid sequence from the active site of TK1 and the other of the first and second antibody or the first and second fragment has specificity for a peptide consisting of an amino acid sequence from the C-terminal region of TK1.

In another embodiment, one of the first and the second antibody or the first and second fragment has specificity for a peptide consisting of a first amino acid sequence from the C-terminal region of TK1 and the other of the first and the second antibody or the first and second fragment has specificity for a peptide consisting of the first amino acid sequence from the C-terminal region of TK1 or a second, different amino acid sequence from the C-terminal region of TK1.

In a further embodiment, one of the first and the second antibody or the first and second fragment has specificity for a peptide consisting of a first amino acid sequence from the C-terminal region of TK1 and the other of the first and the second antibody or the first and second fragment has specificity for a conformation dependent epitope of human TK1.

A fragment of an antibody as used herein can be selected from a group consisting of a single chain antibody, a Fv fragment, a scFv fragment, a Fab fragment, a F(ab′)₂ fragment, a Fab′ fragment, a Fd fragment, a single-domain antibody (sdAb), a scFv-Fc fragment, a di-scFv fragment and a CDR region.

In an embodiment, the kit is a sandwich assay kit. In a particular embodiment, the kit is an Enzyme-Linked Immunosorbent Assay (ELISA) kit and preferably a sandwich ELISA.

In the discussion below, the first antibody or first fragment is assumed to be the capture antibody with the second antibody or second fragment acting as detection antibody. The embodiments are, however, not limited thereto but could switch capture and detection antibodies.

A sandwich ELISA can be used to detect STK1 material in a body sample by preparing a surface of a support, such as a solid support, to which the first antibody or the first fragment is bound as so-called capture antibody. In a preferred embodiment, a known quantity of the first antibody or the first fragment is bound to the surface of the support. Any non-specific binding sites on the surface are optionally, but preferably, blocked. The body sample is then applied to the surface so that any STK1 material present therein will be captured by the immobilized first antibodies or first fragments. Unbound material is preferably removed by one or multiple washing steps. The second antibody or second fragment, typically denoted detection antibody, is then added and is allowed to bind to any STK1 material captured by the first antibody or the first fragment.

The amount of bound second antibody or second fragment is then determined by direct or indirect detection methods. For instance, a label or enzyme can be attached directly to the second antibody or the second fragment or indirectly via a link, such as a biotin-streptavidin or a biotin-avidin link. It is, alternatively, possible to use a secondary antibody or second fragment that is labeled or connected to an enzyme and binds specifically to the second antibody or second fragment.

Hence, in an embodiment the second antibody or second fragment has a covalently attached biotin. Alternatively, the second antibody or second fragment has a covalently attached streptavidin or avidin.

The kit preferably also comprises a horseradish peroxidase (HRP) labeled streptavidin or a HRP labeled avidin. Alternatively, the kit also comprises a HRP labeled biotin. The kit also comprises a HRP substrate, such as a 3,3′,5,5′-tetramethylbenzidine (TMB) substrate, a 3,3′-diaminobenzidine (DAB) substrate or a 2,2′-azino-bis(3-ethylbenzothiazoline-6-sulphonic acid) (ABTS) substrate. In such a case, the level of STK1 material in the sample can be determined by spectrophotometric methods that detect the conversion of the chromogenic substrate by HRP into a colored product that is detectable.

In an embodiment, the kit also comprises a microtiter plate (MCP) as the support to which the first antibody or first fragment is immobilized or is intended to be immobilized.

The kit does not necessarily have to be an ELISA kit. In another embodiment, the kit uses affinity chromatography where the first antibody is bound to the stationary phase, such as to a gel matrix or beads in a column. For instance, the gel matrix or beads could be made of agarose, such as SEPHAROSE®.

In such a case, TK1 material present in a body sample will be entrapped in the column through binding to the immobilized first antibodies or first fragments. Following washing, the bound STK1 material can be eluted and detected using the second antibody or second fragment. For instance, the amount of eluted STK1 material can be determined using Western blotting and with the second antibody or second fragment for STK1 detection using direct or indirect detection methods.

The support could alternatively be magnetic beads, such as DYNABEADS® magnetic beads.

In a further embodiment, the kit is a chemiluminescence immunoassay (CLIA) kit. CLIA is an immunoassay technique where the label is a luminescent molecule. CLIA methods can be direct, using luminophore markers, or indirect, using enzyme markers. Either method may be competitive or non-competitive. In direct CLIA methods, the luminophore markers used are typically acridinium and ruthenium esters, while the enzymatic markers used in indirect methods are typically alkaline phosphatase with adamantyl 1,2-dioxetane aryl phosphate (AMPPD) substrate and HRP with luminol or its derivatives as substrate.

The kit does not necessarily have to include two antibodies or fragment but could instead include only one type of antibodies or fragments.

Furthermore, the kit does not necessarily have to comprise a so-called capture antibody or fragment. In clear contrast, multiple, i.e., at least two, different antibodies or fragments could be used to determine the level of STK1 material without the need for immobilizing at least one of the antibodies or fragments.

In an embodiment, the method also comprises selecting an anti-cancer treatment for the patient based on the predicted survival of the patient. Thus, an optimal or at least suitable anti-cancer treatment is selected for prostate cancer patient based on the determined level of STK1 material in the body sample and thereby based on the predicted survival estimated for the patient based on the determined level of STK1 material in the body sample. This means that patients with a high determined level of STK1 material and thereby a predicted poor survival rate could be selected for a more aggressive anti-cancer treatment as compared to patients with a comparatively lower level of STK1 material and thereby a better predicted survival rate. Examples of anti-cancer treatments that can be selected include one or more of prostatectomy, such as radical prostatectomy, robotic prostatectomy, laparoscopic prostatectomy, or bilateral orchiectomy; radiation therapy, such as external beam radiation therapy, brachytherapy, intensity-modulated radiation therapy (IMRT) or proton therapy; focal therapy, such as cryosurgery or high-intensity focused ultrasound (HIFU) therapy; androgen deprivation therapy (ADT), such as bilateral orchiectomy, luteinizing hormone-releasing hormone (LHRH) agonist therapy, LHRH antagonist therapy or anti-androgen therapy. For instance, prostate cancer patients having a determined level of STK1 material exceeding the previously mentioned threshold value could be selected for a first anti-cancer treatment, whereas other patients with a determined level of STK1 material below the threshold value are selected for a second, different anti-cancer treatment.

In an embodiment, the method comprises selecting a patient surveillance schedule for the patient based on the predicted survival of the patient. Thus, an optimal or at least suitable patient surveillance schedule or scheme is selected for prostate cancer patient based on the determined level of STK1 material in the body sample and thereby based on the predicted survival estimated for the patient based on the determined level of STK1 material in the body sample. This means that patients with a high determined level of STK1 material and thereby a predicted poor survival rate could be selected for a more frequent surveillance and follow-up (first surveillance schedule) as compared to patients with a comparatively lower level of STK1 material and thereby a better predicted survival rate, which instead can follow a less frequent surveillance and follow-up (second surveillance schedule).

### EXAMPLE

The present Example evaluates whether serum TK1 levels could be used as marker for risk of prostate cancer progression after diagnosis.

Materials and Methods

The study population included 40 men with T1 or T2Nx/0M0 prostate cancer at diagnosis and 43 men with de novo metastatic cancer (M1) at diagnosis as confirmed by bone scan imaging. All men were diagnosed and treated at the Tampere University Hospital, Department of Urology between 2000-2010. All men provided serum sample at the time of diagnosis. Information on deaths and causes of death were obtained from national causes of death registry of Statistics Finland.

TK1 levels in serum samples were determined using the AroCell TK210 ELISA (Uppsala, Sweden) according to the manufactures instructions. In brief, serum samples, calibrators and controls were pre-incubated with sample dilution buffer (SDB) for 1 hour at room temperature. After pre-incubation, the samples were added to pre-washed anti-TK1 antibody coated plates and incubated again for 2 hours on a shaking platform. The plates were washed 4 times and biotinylated anti-TK1 antibody was added. After incubation and 4 washes enzyme labeled streptavidin HRP was pipetted into the wells and following an incubation step and 4 washes, a substrate was added. Color development was subsequently stopped and the intensity of color was measured by a spectrophotometer at 450 nm. The color developed was in proportion to the concentration of TK1 protein in the sample. Serum TK1 protein levels in the samples were calculated by using a calibrator curve and a 4-PL curve fitting program. Each sample was analyzed in duplicate and the mean value expressed as μg/L.

Statistical Analysis

Cox proportional hazard regression method was used to estimate hazard ratios for prostate cancer-specific death and death due to any cause. Time metric was months since prostate cancer diagnosis. Follow-up ended at death, emigration from Finland or the closing date of 31 Aug. 2019, whichever came first. Analysis was adjusted for biopsy ISUP Gleason grade group, TNM stage (T-stage) and PSA at diagnosis.

Recurrence-free survival was evaluated in a separate Cox regression model with disease recurrence as the end-point. In this analysis follow-up ended at the date of the first prostate cancer recurrence, death or the closing date, whichever came first. The same model adjustments were used in the analyses on risk of prostate cancer-specific death and death due to any cause.

Study participants where stratified by median and by quartiles of TK1 level. Participants with a TK1 level at or below the median were used as the reference group. Survival trends were analyzed including TK1 level in a Cox regression model as continuous variable. Additionally, Kaplan-Meier curves were used for visual evaluation of the survival differences.

Random forest classification was used to evaluate additional value of TK1 over established prognostic factors as a predictor of prostate cancer death. Different combinations of established prognostic factors ISUP Gleason grade group, PSA at diagnosis, and clinical T-stage, and the novel marker TK1 were used as classifiers for prostate cancer death. Predictive values of the set of prognostic factors were ranked by the classification error per model, where lower classification error indicated better model prediction. Each random forest model was repeated 1000 times for empirical confidence interval estimation using percentile method for the obtained 1000 error estimates, and the median of these as the point estimate for the classification error.

Results

M1 cases were older than M0 cases; median age 72 vs 62 years, respectively. M1 cases were also more often first detected by symptoms (41.9% vs. 12.5%) rather than through prostate-specific antigen (PSA) testing. M1 cases were mainly managed with androgen deprivation therapy, whereas M0 cases were most often managed with radical prostatectomy, see Table 2.

TABLE 2
TK1 levels and population characteristics
by tumor M-stage at diagnosis

| | Prostate cancer M-stage | | | |
|---|---|---|---|---|
| | M0 | | M1 | |
| n of men | 40 | | 43 | |
| Serum TK1 level at | 0.23 | (0.15-0.41) | 0.61 | (0.33-0.82) |
| diagnosis (μg/l); | | | | |
| median (IQR*) | | | | |
| P for difference | <0.001 | | | |
| Age at diagnosis (yrs); | 62 | (57-65) | 72 | (66-76) |
| median (IQR*) | | | | |
| Cancer other than | 3 | (7.5%) | 12 | (27.9%) |
| prostate cancer; n (%) | | | | |
| Prostate volume (ml); | 35 | (28.7-40) | 39 | (28-46) |
| median (IQR*) | | | | |

Prostate cancer clinical characteristics

| Detection method; n (%): | | | | |
|---|---|---|---|---|
| PSA only | 35 | (87.5%) | 23 | (53.5%) |
| Symptoms | 5 | (12.5%) | 18 | (41.9%) |
| Rectal examination | 0 | | 2 | (4.7%) |
| PSA at diagnosis (ng/ml); | 6.7 | (4.7-8.8) | 50 | (18-240) |
| median (IQR*) | | | | |
| Bone scan imaging; n (%) | 15 | (37.5%) | 43 | (100%) |
| Clinical T-stage at | | | | |
| diagnosis; n (%): | | | | |
| T1 | 23 | (57.5%) | 1 | (2.3%) |
| T2 | 17 | (42.5%) | 7 | (16.3%) |

| | | | |
|---|---|---|---|
| T3 | 0 | 18 | (41.9%) |
| T4 | 0 | 17 | (39.5%) |

Treatment characteristics

| Primary treatment; n (%): | | | | |
|---|---|---|---|---|
| Radical prostatectomy | 35 | (87.5%) | 0 | |
| External beam radiation therapy | 3 | (7.5%) | 2 | (4.7%) |
| Androgen deprivation/ | 1 | (2.5%) | 39 | (90.7%) |
| antiandrogen monotherapy | | | | |
| Other** | 1 | (2.5%) | 1 | (2.3%) |

| | | | |
|---|---|---|---|
| Unknown | 0 | 1 | (2.3%) |

Survival information

| | | | | |
|---|---|---|---|---|
| Follow-up (months); | 120 | (114-128) | 67 | (22-110) |
| median (IQR*) | | | | |
| Deaths; n (%) | 2 | (5.0%) | 36 | (83.7%) |
| Prostate cancer deaths; n (%) | 1 | (2.5%) | 24 | (55.8%) |

| | | | |
|---|---|---|---|
| Cancer death due to other | 0 | 3 | (7.0%) |
| than prostate cancer; n (%) | | | |

*Interquartile range
**Includes brachytherapy, surveillance

TABLE 3
TK1 level by tumor ISUP Gleason grade group at biopsy
ISUP Gleason grade group

| | 1 | 2 | 3 | 4 | 5 | |
|---|---|---|---|---|---|---|
| | TK1; median | TK1; median | TK1; median | TK1; median | TK1; median | P for |
| | (IQR) | (IQR) | (IQR) | (IQR) | (IQR) | trend_{age-adjusted} |
| All cases | 0.22 | 0.30 | 0.48 | 0.50 | 0.61 | 0.007 |
| combined | (0.14-0.41) | (0.19-0.71) | (0.32-0.60) | (0.23-0.88) | (0.23-0.88) | |
| M1 cases | 0.56 | 0.82 | 0.48 | 0.58 | 0.62 | 0.379 |
| | (0.26-0.73) | (0.27-1.67) | (0.34-0.64) | (0.23-0.97) | (0.40-1.23) | |
| M0 cases | 0.19 | 0.25 | 0.46 | 0.48 | 0.15 | 0.190 |
| | (0.13-0.33) | (0.15-0.55) | (0.22-0.53) | (0.32-0.60) | (0.15-0.15) | |

Median serum TK1 levels were significantly higher among M1 cases as compared to M0 cases (p for difference <0.001). In the combined study population, STK1 level was a significant predictor of prostate cancer-specific mortality (HR 8.33, 95% CI 2.05-33.88 for TK1 above the median vs median or below) and overall mortality (HR 5.53, 95% CI 1.93-15.85) after adjustment for established prognostic factors including tumor T-stage, PSA at diagnosis, ISUP Gleason grade group at biopsy and M-stage, see Table 4.

TABLE 4
Overall and cancer-specific survival after prostate cancer diagnosis by TK1 level

| | Prostate cancer death | | Death due to any cause | |
|---|---|---|---|---|
| Serum TK1 level | HR (95% | HR (95% | HR (95% | HR (95% |
| at diagnosis (μg/l) | Cl)_{age-adjusted} | Cl)_{multivariable-adjusted} | Cl)_{age-adjusted} | Cl)_{multivariable-adjusted} |

All cases combined

| 0.347 or below | Reference | | Reference | | Reference | | Reference | |
|---|---|---|---|---|---|---|---|---|
| 0.348 or higher | 8.24 | (2.76-24.63) | 8.33 | (2.05-33.88) | 5.27 | (2.41-11.52) | 5.53 | (1.93-15.85) |

| | | | | |
|---|---|---|---|---|
| P for trend | 0.001 | 0.249 | <0.001 | 0.004 |

M1 cases

| 0.611 or below | Reference | | Reference | | Reference | | Reference | |
|---|---|---|---|---|---|---|---|---|
| 0.612 or higher | 2.47 | (1.05-5.82) | 2.08 | (0.80-5.43) | 3.08 | (1.49-6.36) | 3.27 | (1.41-7.57) |

| | | | | |
|---|---|---|---|---|
| P for trend | 0.043 | 0.241 | <0.001 | 0.005 |

Multivariable adjusted estimates were calculated using Cox regression model with adjustment for age and PSA at diagnosis, clinical T-stage and M-stage.

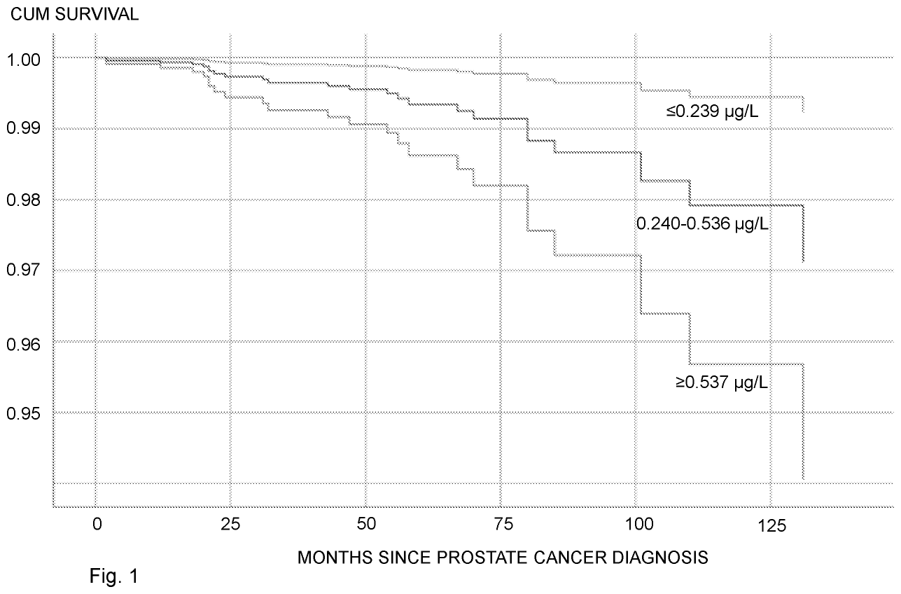

In Kaplan-Meier analysis, Kaplan-Maier cumulative (cum) survival curves start to differ by their TK1 level within 24 months from prostate cancer diagnosis, see FIG. 1.

Population Characteristics

Among men with de novo metastatic disease, median age at diagnosis did not markedly differ by TK1 level (Table 5). Median prostate volume was highest in the quartile of lowest TK1 (quartile 1), while PSA at diagnosis was highest in the quartile of highest TK1 (quartile 4; median PSA 215.5 ng/ml). Clinical T-stage at diagnosis and biopsy ISUP Gleason grade group were similarly distributed across all categories expect in quartile 4 that were only T3/T4 and mostly in ISUP Gleason grade group 4 or 5. Almost all men (91%) were primarily managed with androgen deprivation therapy.

TABLE 5
Population characteristics and prostate cancer clinical characteristics by quartiles of TK1
level. Study population of 43 men diagnosed with M1 prostate cancer

| | Serum TK1 level at diagnosis | | | | | | | |
|---|---|---|---|---|---|---|---|---|
| | Quartile 1 | | Quartile 2 | | Quartile 3 | | Quartile 4 | |
| n of men | 11 | | 11 | | 11 | | 10 | |
| Serum TK1 level at | 0.24 | (0.19-0.29) | 0.50 | (0.42-0.59) | 0.65 | (0.63-0.72) | 1.31 | (0.88-1.98) |
| diagnosis (μg/l); | | | | | | | | |
| median (IQR) | | | | | | | | |
| Age at diagnosis (yrs); | 74 | (68-79) | 68 | (61-74) | 73 | (63-77) | 73 | (67-75) |

| median (IQR) | | | | | | | |
|---|---|---|---|---|---|---|---|
| Prostate volume (ml); | 41 | (39-41) | 28 | (27-28) | — | 28 | (28-28) |

median (IQR)

Prostate cancer clinical characteristics

| PSA at diagnosis (ng/ml); | 49 | (17.7-103) | 29.7 | (16.7-71.8) | 58.10 | (17.2-454) | 215.5 | (37.1-1,259.8) |
|---|---|---|---|---|---|---|---|---|
| median (IQR) | | | | | | | | |
| Clinical T-stage at | | | | | | | | |
| diagnosis; n (%): | | | | | | | | |
| T1 | 1 | (9.1%) | 0 | | 0 | | 0 | |
| T2 | 5 | (45.5%) | 1 | (9.1%) | 1 | (9.1%) | 0 | |
| T3 | 2 | (18.2%) | 6 | (54.5%) | 7 | (63.6%) | 3 | (30%) |
| T4 | 3 | (27.3%) | 4 | (36.4%) | 3 | (27.3%) | 7 | (70%) |
| ISUP Gleason grade | | | | | | | | |
| group; n (%): | | | | | | | | |
| 1 | 2 | (18.2%) | 1 | (9.1%) | 3 | (27.3%) | 0 | |
| 2 | 2 | (18.2%) | 0 | | 1 | (9.1%) | 2 | (20%) |
| 3 | 2 | (18.2%) | 5 | (45.5%) | 3 | (27.3%) | 0 | |
| 4 | 2 | (18.2%) | 1 | (9.1%) | 1 | (9.1%) | 2 | (20%) |
| 5 | 3 | (27.3%) | 4 | (36.4%) | 3 | (27.3%) | 6 | (60%) |

Treatment characteristics

| | | | | | | | | |
|---|---|---|---|---|---|---|---|---|
| Primary treatment; n (%): | | | | | | | | |
| Androgen deprivation | 11 | (100%) | 8 | (72.7%) | 11 | (100%) | 9 | (90%) |

| therapy | | | | | |
|---|---|---|---|---|---|
| External beam | 0 | 2 | (18.2%) | 0 | 0 |

| radiation therapy | | | | | |
|---|---|---|---|---|---|
| Watchful waiting | 0 | 0 | 0 | 1 | (10%) |

| | | | | | |
|---|---|---|---|---|---|
| Unknown | 0 | 1 | (9.1%) | 0 | 0 |

Survival information

| Follow-up (months); | 113 | (85-131) | 80 | (21-129) | 57 | (21-86) | 28 | (6-58) |
|---|---|---|---|---|---|---|---|---|
| median (IQR) | | | | | | | | |
| Deaths; n (%) | 8 | (72.7%) | 8 | (72.7%) | 10 | (90.9%) | 10 | (100%) |
| Prostate cancer | 4 | (36.4%) | 7 | (63.6%) | 6 | (54.5%) | 7 | (70%) |

| deaths; n (%) | | | | | | | |
|---|---|---|---|---|---|---|---|
| Cancer death due to | 0 | 1 | (9.1%) | 1 | (9.1%) | 1 | (10%) |

other than prostate
cancer; n (%)

Serum TK1 Level at Diagnosis as Prognostic Factor for Prostate Cancer-Specific and Overall Survival

Mortality differed by TK1 level; unadjusted prostate cancer-specific mortality was 501/1,000 and 524/1,000 for TK1 at median or below versus above the median, respectively.

Having serum TK1 level above the median at prostate cancer diagnosis was associated with worse prostate cancer-specific (age-adjusted HR 2.47 95% CI 1.05-5.82) and overall survival (HR 3.08, 95% CI 1.49-6.36) compared to men with TK1 level at median or below (Table 6). The risk association remained unchanged after adjustment for tumor Gleason grade group, PSA level at diagnosis or clinical T-stage. A statistically significant increasing risk trend by TK1 level was observed both for prostate cancer-specific and overall risk of death (Table 6).

TABLE 6
Overall and cancer-specific survival after prostate cancer (PCa) diagnosis stratified by
TK1 median at diagnosis. Study population of 43 men with de novo M1 prostate cancer at
diagnosis, i.e. disease diagnosed at metastatic stage, no previous prostate cancer treatments

| Serum TK1 | n of | | | | | | | | |
|---|---|---|---|---|---|---|---|---|---|
| level at | men/ | | | | | | | | |
| diagnosis | PCa | Prostate cancer death | | | | | | | |
| (μg/l) | deaths | HR 1 (95% Cl) | | HR 2 (95% Cl) | | HR 3 (95% Cl) | | HR 4 (95% Cl) | |
| 0.611 or below | 22/11 | Reference | | Reference | | Reference | | Reference | |
| 0.612 or higher | 21/13 | 2.47 | (1.05-5.82) | 2.37 | (1.00-5.66) | 2.32 | (1.00-5.43) | 2.53 | (1.01-6.32) |
| P for trend | | 0.001 | | 0.001 | | 0.015 | | 0.003 | |
| Serum TK1 | | | | | | | | | |
| level at | n of | | | | | | | | |
| diagnosis | men/ | Death due to any cause | | | | | | | |
| (μg/l) | deaths | HR 1 (95% Cl) | | HR 2 (95% Cl) | | HR 3 (95% Cl) | | HR 4 (95% Cl) | |
| 0.611 or below | 22/16 | Reference | | Reference | | Reference | | Reference | |
| 0.612 or higher | 21/20 | 3.08 | (1.49-6.36) | 3.15 | (1.52-6.54) | 2.88 | (1.40-5.89) | 3.77 | (1.69-8.39) |

| | | | | |
|---|---|---|---|---|
| P for trend | <0.001 | <0.001 | 0.002 | <0.001 |

HR 1 = age-adjusted, HR 2 = adjusted for ISUP Gleason grade group, HR 3 = adjusted for PSA at diagnosis, HR 4 = adjusted for clinical T-stage

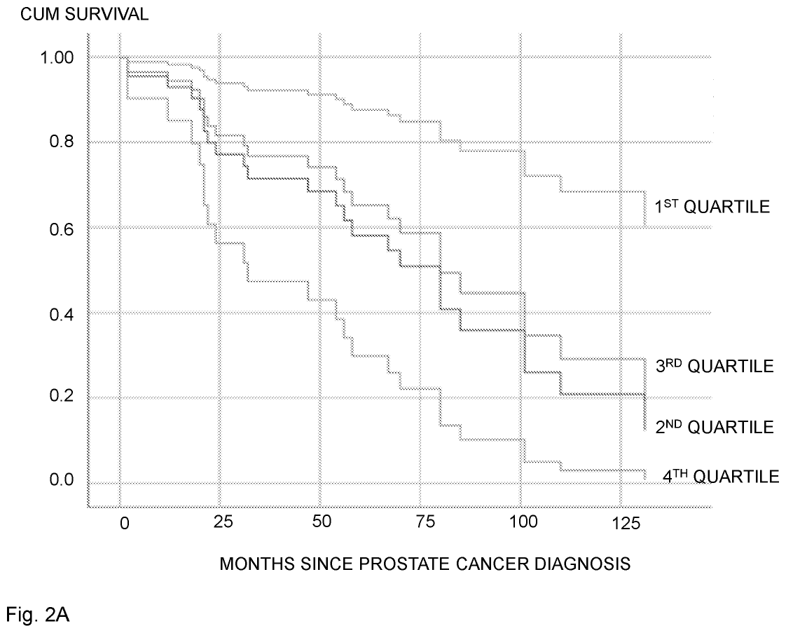

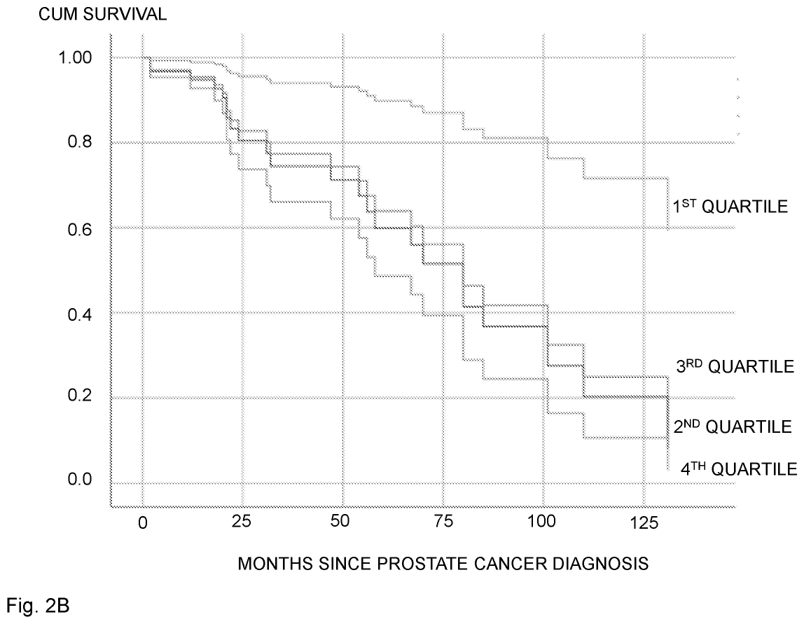

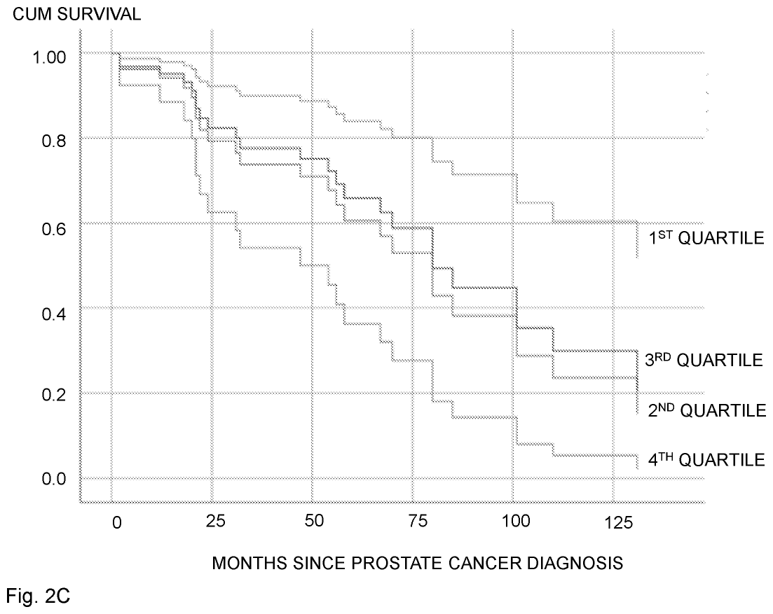

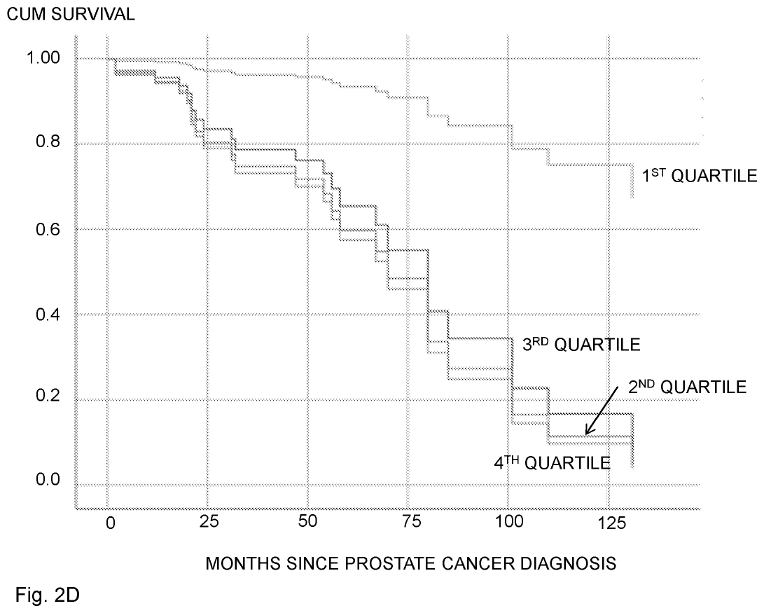

In Kaplan-Meier analysis, stratification by TK1 quartiles showed distinct groups of differing disease-specific survival especially in analyses adjusted for age (FIG. 2A) and PSA at diagnosis (FIG. 2C). Adjustment for ISUP Gleason grade group and clinical T-stage at diagnosis attenuated the survival difference between the top three quartiles, while the quartile with lowest TK1 level still had distinctly better survival compared to men with higher TK1 (FIGS. 2B and 2D). With all model adjustments the survival curves started to diverge by TK1 level clearly approximately within 24 months after prostate cancer diagnosis.

Additive Prognostic Value of TK1 Over Established Prostate Cancer Prognostic Markers

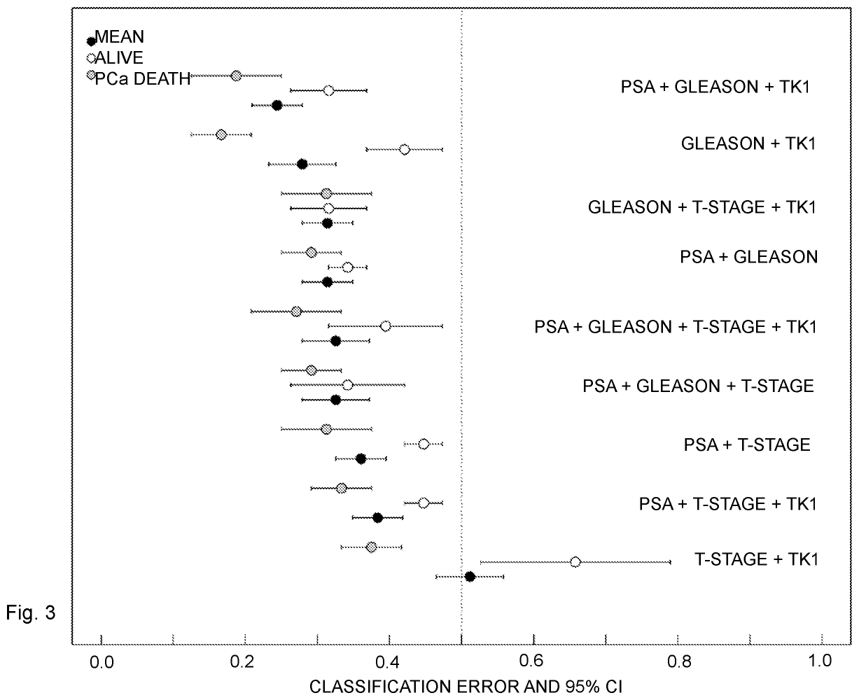

Inclusion of TK1, ISUP Gleason grade group, and PSA as classifiers in the random forest classification model produced most accurate prediction for prostate cancer death compared to any other model (FIG. 3). Notably, combination of these three variables displayed higher mean prediction accuracy compared to model using PSA and ISUP Gleason grade group alone, confirming independent additional prognostic value of TK1. All models with clinical T-stage as a classifier had worse mean prediction error compared to models without it. Therefore T-stage was left out of the models. Overall, the mean prediction error for prostate cancer death using TK1, ISUP Gleason grade group, and PSA in combination is excellent (FIG. 3).

TK1 Among Men with Localized Prostate Cancer at Diagnosis

Of the 40 men with clinically localized cancer at diagnosis, only 1 men died of prostate cancer during median follow-up of 120 months after diagnosis. Median serum TK1 level was considerably lower compared to men with de novo metastatic disease (median 0.61 vs 0.28 μg/l for metastatic and localized cases, respectively). In this group, TK1 level did not markedly vary by biopsy ISUP Gleason grade group (median 0.23 vs. 0.21 μg/l for grade 3-5 vs grade 1-2 tumors, respectively).

In this clinical study of 43 men diagnosed with de novo metastatic prostate cancer, serum TK1 level demonstrated independent prognostic value for both disease-specific and overall survival, improving prognostic accuracy when combined with established clinical factors ISUP Gleason grade group. Therefore TK1 could be used as a marker for risk stratification in this high-risk patient group.

Adjustment for tumor clinical factors affected the risk association differently; very low TK1 level remained associated with good survival with all model adjustments, but adjustment for ISUP Gleason grade group and PSA at diagnosis attenuated the survival differences between the top three quartiles of TK1 level.

In all analyses, the survival curves diverged between TK1 quartiles within 24 months after diagnosis. Cornerstone of treatment in metastatic prostate cancer is androgen deprivation therapy (ADT). In metastatic prostate cancer, median time from ADT initiation to development of castration resistance is 36-42 months. Therefore the findings indicate that ADT is initially effective regardless of TK1 level, but men with high TK1 may develop castration resistance sooner, therefore having poorer survival compared to men with lower TK1.

TK1 also predicted overall survival. Possible explanation is that men with high TK1 at diagnosis have high-volume, aggressive disease bound to progress quickly. Such men are probably at elevated risk of dying of cancer-related complications, such as thromboembolism, which would be reflected in the overall survival.

TK1 level in the serum depends on total number of malignant cells, proportion of malignant cells in the S-phase, i.e., in active proliferation, number of cellular disruptions resulting in leakage, leakage due to other reasons and blood supply. In this study, blood samples for TK1 measurement were drawn at diagnosis, before any cytotoxic treatment had commenced. Therefore, in our case the STK1 level mainly reflects number of malignant cells in active proliferation and cell turnover. Therefore, in treatment-naïve situation TK1 could be used as prognostic marker for metastatic prostate cancer, which could be used to aid risk stratification and treatment selection.

Median TK1 level was lower among men with localized prostate cancer at diagnosis as compared to men with metastatic disease, which also supports TK1 level being affected mostly by number of malignant cells in active proliferation and cell turnover. In this group only one man died of prostate cancer, thus, prognostic value of TK1 could not be evaluated. TK1 measurement is likely to provide added clinical value mainly in cases with advanced disease.

Compared to circulating tumor DNA (ctDNA), another blood-borne marker suggested to have prognostic value, measurement of TK1 protein level is logistically easier as it can be done from stored blood samples and with a simple ELISA kit assay.

In conclusion, the prospective cohort study of 43 men with de novo metastatic prostate cancer shows that TK1 could be used as a new prognostic marker in advanced prostate cancer, accurately predicting disease-specific survival with added value over established clinical markers ISUP Gleason grade group (or Gleason score), T-stage and PSA level.

The proliferation marker TK1 in the serum robustly predicted survival after prostate cancer diagnosis, demonstrating additional value over established clinical risk factors. TK1 should be incorporated in prostate cancer risk stratification when selecting optimal treatment and surveillance schedules for prostate cancer patients.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

1.-27. (canceled)

28. A method for predicting patient survival comprising:
determining a level of serum thymidine kinase 1 (STK1) material in a body sample from a patient diagnosed with prostate cancer using an antibody or a fragment thereof specifically binding to a serum form of human TK1; and
predicting survival of the patient based on the determined level of STK1 material in the body sample.

29. The method according to claim 28, wherein
determining the level of STK1 material comprises determining the level of STK1 material in a serum sample or a plasma sample using the antibody or the fragment thereof specifically binding to the serum form of human TK1; and
predicting survival of the patient comprises predicting survival of the patient based on the determined level of STK1 material in the serum sample or the plasma sample.

30. The method according to claim 28, wherein determining the level of STK1 material in the body sample comprises:
contacting the body sample with the antibody or the fragment thereof; and
measuring an amount of antibody or fragment thereof bound to the STK1 material.

31. The method according to claim 30, further comprising correlating the measured amount of antibody or fragment thereof bound to the STK1 material to a level of STK1 material.

32. The method according to claim 31, wherein correlating the measured amount of antibody or fragment comprises correlating the measured amount of antibody or fragment thereof to a level of STK1 material using a pre-defined correlation between measured amount of antibody or fragment thereof bound to recombinant human TK1 and a concentration of recombinant human TK1.

33. The method according to claim 28, wherein determining the level of STK1 material comprises determining, using the antibody or the fragment thereof specifically binding to the serum form of human TK1, the level of STK1 material in the body sample taken from the patient in connection with diagnosing the patient with prostate cancer.

34. The method according to claim 28, wherein determining the level of STK1 material comprises determining the level of STK1 material in the body sample from a patient diagnosed with metastatic prostate cancer using the antibody or the fragment thereof specifically binding to the serum form of human TK1.

35. The method according to claim 28, further comprising estimating a hazard ratio (HR) for the patient based on the determined level of STK1 material in the body sample, wherein predicting survival of the patient comprises predicting survival of the patient based on the estimated HR.

36. The method according to claim 28, further comprising comparing the determined level of STK1 material in the body sample with a threshold value, wherein predicting survival of the patient comprises predicting poor survival of the patient if the determined level of STK1 material in the body sample exceeds the threshold value and otherwise predicting good survival of the patient.

37. The method according to claim 36, wherein the threshold value is selected within an interval of from 0.25 to 0.75 μg/L.

38. The method according to claim 37, wherein the threshold value is selected within an interval of from 0.3 to 0.65 μg/L.

39. The method according to claim 38, wherein the threshold value is selected within an interval of from 0.325 and 0.375 μg/L.

40. The method according to claim 36, wherein the threshold value is selected within an interval of from 0.15 to 0.45 μg/L.

41. The method according to claim 40, wherein the threshold value is selected within an interval of from 0.175 to 0.40 μg/L.

42. The method according to claim 41, wherein the threshold value is selected within an interval of from 0.20 and 0.30 μg/L.

43. The method according to claim 42, wherein the threshold value is selected within an interval of from 0.20 and 0.25 μg/L.

44. The method according to claim 36, wherein the threshold value is selected within an interval of from 0.30 to 0.90 μg/L.

45. The method according to claim 44, wherein the threshold value is selected within an interval of from 0.40 to 0.80 μg/L.

46. The method according to claim 45, wherein the threshold value is selected within an interval of from 0.50 and 0.70 μg/L.

47. The method according to claim 46, wherein the threshold value is selected within an interval of from 0.60 and 0.65 μg/L.

48. The method according to claim 36, wherein
comparing the determined level of STK1 material comprises comparing the determined level of STK1 material in the body sample with a first threshold value and a second threshold value;
predicting survival of the patient comprises predicting a good survival rate if the determined level of STK1 material in the body sample is below, or equal to or below, the first threshold value, predicting an intermediate survival rate if the determined level of STK1 material in the body sample is equal to or above, or above, the first threshold value but below, or equal to or below, the second threshold value and predicting a poor survival rate if the determined level of STK1 material in the body sample is equal to or above, or above, the second threshold value.

49. The method according to claim 48, wherein
the first threshold value is selected within an interval of from 0.15 μg/L to 0.45 μg/L; and
the second threshold value is selected within an interval of from 0.30 μg/L to 0.90 μg/L, with the proviso that the second threshold value is larger than the first threshold value.

50. The method according to claim 49, wherein
the first threshold value is selected within an interval of from 0.175 μg/L to 0.40 μg/L; and
the second threshold value is selected within an interval of from 0.40 μg/L to 0.80 μg/L, with the proviso that the second threshold value is larger than the first threshold value.

51. The method according to claim 50, wherein
the first threshold value is selected within an interval of from 0.20 μg/L to 0.30 μg/L; and
the second threshold value is selected within an interval of from 0.45 μg/L to 0.70 μg/L.

52. The method according to claim 51, wherein
the first threshold value is selected within an interval of from 0.225 μg/L to 0.25 μg/L; and
the second threshold value is selected within an interval of from 0.50 μg/L to 0.60 μg/L.

53. The method according to claim 28, further comprising determining prostate-specific antigen (PSA) in a body sample from the patient, wherein predicting survival of the patient comprises predicting survival of the patient based on the determined level of STK1 material in the body sample and based on the determined PSA in the body sample.

54. The method according to claim 28, further comprising determining International Society of Urological Pathology (ISUP) Gleason grade group or Gleason score for the patient, wherein predicting survival of the patient comprises predicting survival of the patient based on the determined level of STK1 material in the body sample and based on the determined ISUP Gleason grade group or Gleason score.

55. The method according to claim 54, further comprising determining prostate-specific antigen (PSA) in a body sample from the patient, wherein predicting survival of the patient comprises predicting survival of the patient based on the determined level of STK1 material in the body sample, based on the determined PSA in the body sample and based on the determined ISUP Gleason grade group or Gleason score.

56. The method according to claim 28, wherein the antibody or the fragment thereof is a monoclonal antibody or a fragment specifically binding to the serum form of human TK1.

57. The method according to claim 56, wherein the monoclonal antibody or the fragment thereof is selected from the group consisting of:
a monoclonal antibody or a fragment thereof having specificity for GEAVAARKLF (SEQ ID NO: 1) of human TK1;
a monoclonal antibody or a fragment thereof having specificity for at least one of NCPVPGKPGE (SEQ ID NO: 2), PVPGKPGEAV (SEQ ID NO: 3) and NCPVPGKPGEAV (SEQ ID NO: 4) of human TK1; and
a monoclonal antibody or a fragment thereof having specificity for a conformation dependent epitope of human TK1.

58. The method according to claim 57, wherein the monoclonal antibody or the fragment thereof has
a variable heavy (VH) domain complementarity determining region 1 (CDR1) having amino acid sequence SEQ ID NO: 5;
a VH domain CDR2 having amino acid sequence SEQ ID NO: 6;
a VH domain CDR3 having amino acid sequence SEQ ID NO: 7;
a variable light (VL) domain CDR1 having amino acid sequence SEQ ID NO: 8;
a VL domain CDR2 having amino acid sequence SEQ ID NO: 9; and
a VL domain CDR3 having amino acid sequence SEQ ID NO: 10.

59. The method according to claim 57, wherein the monoclonal antibody or the fragment thereof has
a variable heavy (VH) domain complementarity determining region 1 (CDR1) having amino acid sequence SEQ ID NO: 5;
a VH domain CDR2 having amino acid sequence SEQ ID NO: 11;
a VH domain CDR3 having amino acid sequence SEQ ID NO: 12;
a variable light (VL) domain CDR1 having amino acid sequence SEQ ID NO: 13;
a VL domain CDR2 having amino acid sequence SEQ ID NO: 9; and
a VL domain CDR3 having amino acid sequence SEQ ID NO: 10.

60. The method according to claim 57, wherein the monoclonal antibody or the fragment thereof has
a variable heavy (VH) domain complementarity determining region 1 (CDR1) having amino acid sequence SEQ ID NO: 14;
a VH domain CDR2 having amino acid sequence SEQ ID NO: 15;
a VH domain CDR3 having amino acid sequence SEQ ID NO: 16;
a variable light (VL) domain CDR1 having amino acid sequence SEQ ID NO: 17;
a VL domain CDR2 having amino acid sequence SEQ ID NO: 18; and
a VL domain CDR3 having amino acid sequence SEQ ID NO: 19.

61. The method according to claim 56, wherein determining the level of STK1 material comprises determining the level of STK1 material in the body sample using a kit for determining a level of STK1 material in a body sample comprising:
a first monoclonal antibody or a first fragment thereof having specificity for an epitope selected from the group consisting of:
GEAVAARKLF (SEQ ID NO: 1) of human TK1;
at least one of NCPVPGKPGE (SEQ ID NO: 2), PVPGKPGEAV (SEQ ID NO: 3) and NCPVPGKPGEAV (SEQ ID NO: 4) of human TK1; and
a conformation dependent epitope of human TK1; and
a second monoclonal antibody or a second fragment thereof having specificity for an epitope selected from the group consisting of:
GEAVAARKLF (SEQ ID NO: 1) of human TK1;
at least one of NCPVPGKPGE (SEQ ID NO: 2), PVPGKPGEAV (SEQ ID NO: 3) and NCPVPGKPGEAV (SEQ ID NO: 4) of human TK1; and
a conformation dependent epitope of human TK1.

62. The method according to claim 61, wherein one of the first monoclonal antibody or first fragment thereof and the second monoclonal antibody or second fragment thereof is immobilized to a solid support or intended to be immobilized to the solid support.

63. The method according to claim 61, wherein the kit is an Enzyme-Linked Immunosorbent Assay (ELISA) kit.

64. The method according to claim 28, further comprising selecting an anti-cancer treatment for the patient based on the predicted survival of the patient.

65. The method according to claim 28, further comprising selecting a patient surveillance schedule for the patient based on the predicted survival of the patient.


SEQUENCE LISTING (28 sequences)

SEQ ID NO: 1 - protein, 10 aa, Homo sapiens
GEAVAARKLF

SEQ ID NO: 2 - protein, 10 aa, Homo sapiens
NCPVPGKPGE

SEQ ID NO: 3 - protein, 10 aa, Homo sapiens
PVPGKPGEAV

SEQ ID NO: 4 - protein, 12 aa, Homo sapiens
NCPVPGKPGEAV

SEQ ID NO: 5 - protein, 5 aa, Mus musculus
DYEMH

SEQ ID NO: 6 - protein, 17 aa, Mus musculus
AIHPGYGGTAYNQKFKG

SEQ ID NO: 7 - protein, 7 aa, Mus musculus
FITKFDY

SEQ ID NO: 8 - protein, 16 aa, Mus musculus
KSSQSLLDSDGKTFLN

SEQ ID NO: 9 - protein, 7 aa, Mus musculus
LVSKLDS

SEQ ID NO: 10 - protein, 9 aa, Mus musculus
WQGTHFPWT

SEQ ID NO: 11 - protein, 17 aa, Mus musculus
AILPGSGGTAYNQKFKG

SEQ ID NO: 12 - protein, 7 aa, Mus musculus
LITTFDY

SEQ ID NO: 13 - protein, 16 aa, Mus musculus
KSSQSLLDSDGKTYLN

SEQ ID NO: 14 - protein, 6 aa, Mus musculus
SGYSWH

SEQ ID NO: 15 - protein, 16 aa, Mus musculus
YIHYSGSTTYNPSLKG

SEQ ID NO: 16 - protein, 10 aa, Mus musculus
WGTGHWYFDV

SEQ ID NO: 17 - protein, 14 aa, Mus musculus
RSSTGAVTTTNYAN

SEQ ID NO: 18 - protein, 7 aa, Mus musculus
GTNNRVP

SEQ ID NO: 19 - protein, 9 aa, Mus musculus
ALWYSNHWV

SEQ ID NO: 20 - protein, 15 aa, Homo sapiens
KPGEAVAARKLFAPQ

SEQ ID NO: 21 - protein, 23 aa, Homo sapiens
AYTKRLGTEKEVEVIGGADKYHS

SEQ ID NO: 22 - protein, 185 aa, Artificial Sequence
  rabbit mAb 6C6 VH domain
METGLRWLLLVAVLKGVQCQEQLEESGGDLVKPEGSLTLTCTASRFSFSSSYWICWVRQAPGKGLEWIACIYAGDSGSSYYASWAKGRFTVSKTSSTTVTLQTTSLTAADTATYFCARASVGAAYDYFALWGPGTLVTVSSGQPKAPSVFPLAPCCGDTPSSTVTLGCLVKGYLPEPVTVTWNSG

SEQ ID NO: 23 - protein, 236 aa, Artificial Sequence
  rabbit mAb 6C6 VL domain
MDTRAPTQLLGLLLLWLPGARCALVMTQTPASVEAAMGGTVTIKCQASEDVSSHLAWYQQRPGQPPKLLIYGASDLASGVPSRFTGSGSGTQFTLAISDLECADAATYYCQGYYYISDSPYVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 24 - protein, 187 aa, Artificial Sequence
  rabbit 4H4 VH domain
METGLRWLLLVAVLKGVQCQSLEESGGGLVQPEGSLTLTCTASGFSFSSGYDMCWVRQTPGKGLEWIACISVDSDGVTYYASWAKGRFTISKTSSTTVTLQMTSLTAADTATYFCARGYESSSGVYIPYFTLWGPGTLVTVSSGQPKAPSVFPLAPCCGDTPSSTVTLGCLVKGYLPEPVTVTWNSG

SEQ ID NO: 25 - protein, 238 aa, Artificial Sequence
  rabbit mAb 4H4 VL domain
MDMRAPTQLLGLLLLWLPGARCADIVLTQTPASVEAAVGGTVTIKCQASQSIYSYLAWYQHKPGQPPKLLIYKASTLASGVPSRFKGSGSGTEYTLTISDLECADAATYYCQHYYYSSTSGGGVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 26 - protein, 179 aa, Artificial Sequence
  rabbit mAb 23C11 VH domain
METGLRWLLLVAVLKGVQCQSLEESGGRLVTPGTPLTLTCTASGFSLSNYYMSWVRQAPGKGLEWIGIIYGDDNTYCANWTKGRFTISKTSTTVDLTITSPTTEDTATYFCARGPDYIAAKMDIWGPGTLVTVSLGQPKAPSVFPLAPCCGDTPSSTVTLGCLVKGYLPEPVTVTWNSG

SEQ ID NO: 27 - protein, 239 aa, Artificial Sequence
  rabbit mAb 23C11 VL domain
MDTRAPTQLLGLLLLWLPGARCDVVMTQTPASVEAAVGGTVTIKCQASQSISGYLSWYQQKPGQRPKLLIYRASTLESGVPSRFKGSGSGTEFTLTISDLECADAATYYCQCTYGSSTFSSYGNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 28 - protein, 234 aa, Homo sapiens
MSCINLPTVLPGSPSKTRGQIQVILGPMFSGKSTELMRRVRRFQIAQYKCLVIKYAKDTRYSSSFCTHDRNTMEALPACLLRDVAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQCSPAN